(19) United States
(12) Patent Application Publication
(10) Pub. No.: US 2003/0007972 A1
(43) Pub. Date: Jan. 9, 2003

(54) Cytokine antagonists and other biologics for the treatment of bone metastases

(76) Inventor: Edward Tobinick, Los Angeles, CA (US)

Correspondence Address: Ezra Sutton, P.A., Plaza 9, 900 Route 9, Woodbridge, NJ, 07095, US

(21) Appl. No.: 10/236,097

(22) Filed: Sep. 6, 2002

Related U.S. Application Data
(63) Continuation-in-part of application No. 09/841,844, filed on Apr. 25, 2001.
Application No. 09/841,844 is a continuation-in-part of application No. 09/826,976, filed on Apr. 5, 2001, now Pat. No. 6,419,944.
Application No. 09/826,976 is a continuation-in-part of application No. 09/563,651, filed on May 2, 2000, now Pat. No. 6,471,961.
Application No. 09/563,651 is a continuation-in-part of application No. 09/476,643, filed on Dec. 31, 1999, now Pat. No. 6,177,077.
Application No. 09/476,643 is a continuation-in-part of application No. 09/275,070, filed on Mar. 23, 1999, now Pat. No. 6,015,557.
Application No. 09/275,070 is a continuation-in-part of application No. 09/256,388, filed on Feb. 24, 1999, now abandoned.

(51) Int. Cl. A61K 39/395; A61K 38/17

(52) U.S. Cl. 424/145.1; 514/12

(57) ABSTRACT

Methods for treating bone metastases in humans by administering a therapeutically effective dose of specific biologics are presented. The biologics of consideration include antagonists of tumor necrosis factor or of interleukin-1; or biologic inhibitors of osteoclastogenesis, including OPG. The administration of these biologics is performed by specific methods, most of which fall into the category of anatomically localized injection designed for perilesional or intralesional use in proximity to the site of tumor metastasis to bone. Anatomically localized administration involving perilesional or intralesional use includes, but is not limited to, subcutaneous, intramuscular, interspinous, epidural, peridural, parenteral or perispinal administration.

### RELATED APPLICATIONS

This is a continuation-in-part of application Ser. No. 09/841,844 filed on Apr. 25, 2001, which is a continuation-in-part of application Ser. No. 09/826,976, filed on Apr. 5, 2001, now U.S. Pat. No. 6,419,944 which is a continuation-in-part of application Ser. No. 09/563,651, filed on May 2, 2000, which is a continuation-in-part of application Ser. No. 09/476,643, filed on Dec. 31, 1999, now U.S. Pat. No. 6,177,077, which is a continuation-in-part of application Ser. No. 09/275,070, filed on Mar. 23, 1999, now U.S. Pat. No. 6,015,557, which is a continuation-in-part of application Ser. No. 09/256,388, filed on Feb. 24, 1999, now abandoned.

### FIELD OF THE INVENTION

The present invention relates to novel methods of use of specific biologics for the treatment of bone metastases in humans. More particularly, these biologics are used in a new treatment of malignant diseases, including, but not limited to cancers, causing destruction of bone, utilizing specific anatomic methods of administration of these specific biologics. The administration of these biologics is performed by specific methods, most, but not all of which fall into the category of anatomically localized injection designed for perilesional or intralesional use. Anatomically localized administration involving perilesional or intralesional use includes, but is not limited to subcutaneous, intramuscular, interspinous, epidural, peridural, parenteral, or perispinal administration. The biologics included are those designed to block the action of, inhibit, or antagonize the biologic effects of tumor necrosis factor or interleukin-1; or those which cause inhibition of osteoclastogenesis, including, but not limited to osteoprotegerin (OPG). These antagonists may take the form of a fusion protein (such as etanercept); a monoclonal antibody (such as infliximab); a binding protein (such as onercept, Serono); an antibody fragment (such as CDP 870, Pharmacia); or other types of molecules which are potent, selective, and specific inhibitors of the action of these pro-inflammatory cytokines and are capable of being used by parenteral injection.

### BACKGROUND OF THE INVENTION

Localized administration for the treatment of localized clinical disorders has many clinical advantages over the use of conventional systemic treatment. Locally administered medication after delivery diffuses through local capillary, venous, arterial, and lymphatic action to reach the anatomic site of bone metastasis.

All of the cytokine antagonists which are currently available have been developed for systemic administration. This is because all were developed to treat systemic illnesses, including rheumatoid arthritis, juvenile rheumatoid arthritis, psoriatic arthritis, or Crohn's Disease.

The use of cytokine antagonists to treat cancer metastatic to bone is discussed in several previous patents of this inventor, including U.S. Pat. Nos. 6,015,557, 6,177,077, 6,419,944 B2 and other pending applications of this inventor. This invention includes further applications of these ideas.

Localized administration, including perilesional or intralesional administration, when compared to systemic administration, carries with it one or more of the following advantages:

1) greater efficacy due to the achievement of higher local concentration;

2) greater efficacy due to the ability of the administered therapeutic molecule to reach the target tissue without degradation caused by hepatic or systemic circulation;

3) more rapid onset of action;

4) longer duration of action; and

5) Potentially fewer side effects, due to lower required dosage.

The inventor's extensive clinical experience utilizing local administration of etanercept for treating lumbar and cervical radiculopathy has demonstrated the dramatic efficacy, and the extraordinarily rapid onset of action produced by perilesional administration of etanercept for these clinical disorders. Perilesional administration of the biologic agents of consideration here, which include etanercept, for use in treating skeletal metastases due to malignancy, share the above advantages.

Pain is a common accompaniment of cancer. Much cancer-related pain is due to bone metastases. Bone destruction from malignancy can be due to either primary bone lesions, as in multiple myeloma, or metastases to bone from cancers with primary lesions distant from the sites of bone destruction. Many cancers can metastasize to bone. The spine and ribs are among the most common sites of metastasis. Both of these sites are amenable to therapeutic intervention utilizing perilesional administration of biologics.

It has been estimated that more than 1.5 million patients with cancer worldwide have bone metastases. Bone metastases are commonly seen in patients with breast, prostate, thyroid, bladder, lung and renal cancer, as well as patients with malignant melanoma and other forms of cancer. Among the most common are breast cancer and prostate cancer, which account for roughly 80% of bone metastases. Of the patients with breast cancer, about 30% will develop bone metastases. These metastases are often painful and often are osteolytic, causing bone destruction which can result in pathologic fractures. Additionally these lesions can cause nerve root or spinal cord compression. Current treatment regimens, including local radiation, biphosphonates, or trastuzumab can be helpful but are not curative.

The therapeutic molecules of consideration here have many biologic effects. Etanercept, for example, in addition to being a potent anti-inflammatory also has important anti-apoptotic effects. TNF antagonists, IL-1 antagonists, and OPG all act to inhibit osteoclastogenesis. Inhibition of osteoclastogenesis is thought to be important for the therapeutic improvement seen when used for patients with inflammatory arthritis. Osteoclastogenesis has also been implicated as a mechanism by which malignant tumors accomplish destruction of bone. These metastatic tumors may cause activation of osteoclasts, thereby leading to bone destruction. Inhibition of tumor induced osteolysis may be accomplished by inhibiting tumor promoted osteoclastogenesis through the administration of the biologics of consideration here.

Biologics have been developed which have been shown to offer dramatic clinical benefit for systemic illnesses in humans, even for those disorders which have not responded to large and repeated doses of corticosteroids. These biologics fall into the category of cytokine antagonists because they block, or antagonize, the biologic action of a specific cytokine which has adverse clinical effects. These cytokines include the pro-inflammatory cytokines interleukin-1 and tumor necrosis factor.

Specific inhibitors of TNF, only recently commercially available, now provide the possibility of therapeutic intervention in TNF mediated disorders. These agents have been developed to treat systemic illnesses, and therefore have been developed for systemic administration. Various biopharmaceutical companies have developed TNF antagonists to treat systemic illnesses: Immunex Corporation developed etanercept (Enbrel) to treat rheumatoid arthritis; Johnson and Johnson developed infliximab (Remicade®) to treat Crohn's Disease and rheumatoid arthritis; D2E7, a human anti-TNF monoclonal antibody (Abbott) is being developed to treat rheumatoid arthritis and Crohn's Disease; Celltech is developing CDP 571 to treat Crohn's Disease and CDP 870 to treat rheumatoid arthritis; and Serono is developing onercept, a recombinant TNF binding protein (r-TBP-1) for treating rheumatoid arthritis and psoriasis/psoriatic arthritis.

Recent research has demonstrated that a new TNF antagonist can be manufactured from an existing molecule by subtracting a portion of the amino acid sequence from the molecule. This has the advantage of making the molecule smaller. This smaller molecule can be easier to manufacture and may have clinical advantages, such as reduced immunogenicity in the human in vivo. Therefore, the molecules of consideration here shall also include, in addition to those specified, any molecule which contains a fragment of any of the named molecules. A fragment shall be defined as an identical amino acid sequence 50% or greater in length of the original molecule and possessing TNF binding capability or interleukin-1 binding capability.

### DESCRIPTION OF THE PRIOR ART

U.S. Pat. No. 5,863,769 discloses using IL-1 RA for treating various diseases. However, it does not disclose administering cytokine antagonists by intralesional or perilesional injection for the treatment of malignant metastases to bone.

U.S. Pat. No. 6,013,253 discloses using interferon and IL-1 RA for treating multiple sclerosis. However, it does not disclose administering cytokine antagonists by intralesional or perilesional injection for the treatment of malignant metastases to bone.

U.S. Pat. No. 5,075,222 discloses the use of IL-1 inhibitors for treatment of various disorders. However, it does not disclose administering cytokine antagonists by intralesional or perilesional injection for the treatment of malignant metastases to bone.

U.S. Pat. No. 6,159,460 discloses the use of IL-1 inhibitors for treatment of various disorders. However, it does not disclose administering IL-1 antagonists by intralesional or perilesional injection for the treatment of malignant metastases to bone.

U.S. Pat. No. 6,096,728 discloses the use of IL-1 inhibitors for treatment of various disorders. However, it does not disclose the use of IL-1 antagonists by intralesional or perilesional administration for the treatment of malignant metastases to bone.

U.S. Pat. No. 6,369,027 discloses the use of osteoprotegerin for treatment of various disorders of bone. However, it does not disclose the use of OPG by intralesional or perilesional administration for the treatment of malignant metastases to bone.

U.S. Pat. No. 6,277,969 discloses the use of anti-TNF antibodies for treatment of various disorders. However, it does not disclose the use of TNF antagonists by intralesional or perilesional administration for the treatment of malignant metastases to bone.

U.S. Pat. No. 5,605,690 discloses the use of TNF inhibitors for treatment of various disorders. However, it does not disclose the use of TNF antagonists by intralesional or perilesional administration for the treatment of malignant metastases to bone.

None of the prior art patents disclose or teach the use of localized administration of a cytokine antagonist as in the present invention for suppression and inhibition of the action of a specific cytokine in a human to treat malignant metastases to bone, in which the cytokine antagonist provides the patient with a better opportunity to heal, slows disease progression, or otherwise improves the patient's health.

Accordingly, it is an object of the present invention to provide a biologic administered through anatomically localized administration as a new method of pharmacologic treatment of malignant metastases to bone; such that the use of these biologics will result in the amelioration of these conditions.

Another object of the present invention is to provide cytokine antagonists for providing suppression and inhibition of the action of specific cytokines in a human to treat malignant metastases to bone.

Another object of the present invention is to provide cytokine antagonists that produce biologic effects in patients with bone metastases by inhibiting the action of specific cytokines in the human body for the immediate, short term (acute conditions) and long term (chronic conditions), such that these biologic effects will produce clinical improvement in the patient and will give the patient a better opportunity to heal, slow disease progression, prevent neurological damage, reduce pain, or otherwise improve the patient's health.

Another object of the present invention is to provide cytokine antagonists, using anatomically localized administration or systemic administration as the preferred forms of administration, that offer acute and chronic treatment regimens for treating malignant bone metastases.

### SUMMARY OF THE INVENTION

The present invention provides methods for treating malignant bone metastases in humans by administering to the human a therapeutically effective dose of a specific biologic. The biologics of consideration include antagonists of tumor necrosis factor or of interleukin-1; or biologic inhibitors of osteoclastogenesis, including OPG. The administration of these biologics is performed by specific methods, most, but not all of which fall into the category of anatomically localized injection designed for perilesional or intralesional use. Anatomically localized administration involving perilesional or intralesional use includes, but is not limited to subcutaneous, intramuscular, interspinous, epidural, peridural, parenteral or perispinal administration.

### DETAILED DESCRIPTION OF THE PREFERRED EMBODIMENTS

Anatomically localized administration is a novel concept for a delivery method for cytokine antagonists for treating malignant bone metastases.

Occasionally intralesional injection into the tumor itself or into bone whose architecture has been damaged by tumor will be possible. Additionally some patients may benefit by systemic administration of the therapeutic agent, particularly those with multiple lesions. For the great majority of the patients with a malignant bone metastasis, however, perilesional administration is the preferred method of delivery. Perilesional is defined by the Miller-Keane Medical Dictionary, 2000 as “located or occurring around a lesion”. The inventor selected this term for use in this invention because it describes the fact that medication need only be delivered to an anatomic area close to the exact area of pathology. The therapeutic molecule, a biologic, then reaches the target tissue by diffusion through surrounding tissue and thereby achieves therapeutic concentration at the site of bone metastasis.

One of the advantages of this method of delivery is that administration is simplified. For example, administration for the treatment of a bone metastasis to the lumbar spine is effective by the interspinous route adjacent to the involved vertebrae. This route is simple and safe. Hemorrhage due to the use of long or large bore needles is minimized because subcutaneous administration, by the perilesional route, requires only a short, narrow bore needle. Time-consuming and difficult epidural injection is not necessary. Local perilesional administration also has the advantage of providing a depot of therapeutic medication in the surrounding tissue, which will provide therapeutic levels of medication to the treatment site for a prolonged period of time. This decreases the necessity for another injection of medication. Additionally, administering medication locally limits the exposure of the medication to the systemic circulation, thereby decreasing renal and hepatic elimination of the medication, and decreasing exposure of the medication to systemic metabolism. All of these factors tend to increase the therapeutic half-life of the administered cytokine antagonist. Taken together, localized anatomic administration carries with it significant clinical advantages over the various forms of systemic administration previously used with these cytokine antagonists. These forms of systemic administration include the intravenous route; the intramuscular route, when the site of intramuscular administration is remote from the site of pathology; the subcutaneous route, when the site of subcutaneous administration is remote from the site of pathology (such as an abdominal, thigh, or arm administration for the treatment of sciatica); or other methods of administration which rely on the use of the systemic circulation to deliver the medication to the target area of pathology.

For the sake of this invention, the following definitions apply: perilesional is defined as in anatomic proximity to the site of the pathologic process being treated; perispinal is defined as in anatomic proximity to the spine; and peridural is defined as in anatomic proximity to the dura of the spinal cord. Perilesional is used generally to indicate that the biologic is administered in close enough anatomic proximity to allow the therapeutic molecules to reach the target area of pathology by local diffusion within a reasonably short period of time. In general, for purposes of this invention, to deliver the therapeutic medication by perilesional administration one would attempt to deliver the medication within 10 centimeters of the tumor in the bone to allow the medication to reach therapeutic concentration within several hours, and in the best case scenario within minutes.

Biologics to be used for the treatment of malignant bone metastases for the purposes of this patent fall into the general categories of TNF antagonists, interleukin-1antagonists, or biologic inhibitors of osteoclastogenesis, including, but not limited to OPG (osteoprotegerin).

TNF antagonists include, but are not limited to the following: etanercept (Enbrel®—Amgen); infliximab (Remicade®—Johnson and Johnson); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R1) (Amgen); and onercept, a recombinant TNF binding protein (r-TBP-1) (Serono). Antagonists of interleukin-1 include, but are not limited to Kineret®(recombinant IL1-RA, Amgen), IL1-Receptor Type 2 (Amgen) and IL-1 Trap (Regeneron).

Pain due to a bone metastasis can be the presenting symptom in patients with cancer, which can then lead to discovery of the primary cancer. Patients with bone metastases require thorough evaluation and treatment of their primary malignancy, and consideration of treatment of bone metastases with all available effective modalities, including radiation, biphosphonates, chemotherapy, surgery, etc. Bone metastases may be localized utilizing radiographs or positron-emission tomography.

Cord compression due to metastatic cancer is a catastrophic event leading to rapid paralysis if not quickly diagnosed and treated. It is most common with cancers of the breast, colon, lung and prostate, but can be a complication of metastatic disease from a wide variety of malignancies, including melanoma and multiple myeloma. Current treatment regimens include high dose steroids, emergency radiation treatment, and/or emergent surgical decompression. Paralysis can occur within hours, so treatment must be initiated within this time period to avoid permanent sequelae.

The emergent use of a biologic, delivered by anatomically localized administration, may ameliorate neurological damage in this clinical setting.

Most patients presenting for treatment of bone metastases will have experienced chronic pain and have been refractory to treatment with conventional therapy. Patients will need to be evaluated for risk factors related to the use of the biologic in consideration. For example, active infection is a contraindication to the use of a biologic TNF antagonist. After consideration of the possible risks of administration of the agent in question, and elimination of any contraindications, the bone metastasis in question must be carefully localized. Radiographs and PET scans previously obtained may be useful, as well as physical examination, which will usually reveal localized areas of tenderness. Metastases to the spine and ribs are usually accessible. The biologic, in a therapeutically effective dose, can then be administered to a site anatomically adjacent to the bone metastasis utilizing a small gauge needle. This can often be done by subcutaneous injection. Spinal metastases are also amenable to interspinous, epidural, peridural, parenteral or perispinal injections. After injection pressure is applied to maximize hemostasis. Patients may often experience rapid pain relief. Treatment as discussed here may also result in cessation of tumor growth or in diminution in the rate of tumor progression due to a direct effect on the tumor or as a result in a change in the tumor microenvironment.

In one preferred embodiment a patient with cancer metastatic to a lumbar vertebrae complaining of severe persistent pain is treated by injection of a TNF antagonist selected from the group of etanercept, infliximab, CDP 870, D2E7, or onercept in a therapeutically effective dose to the anatomic area adjacent to the involved vertebrae.

In another preferred embodiment a patient with cancer metastatic to a lumbar vertebrae complaining of severe persistent pain is treated by injection of a IL-1 antagonist selected from the group of IL-1 RA, Kineret®, IL-1 R type 2 or IL-1 Trap in a therapeutically effective dose to the anatomic area adjacent to the involved vertebrae.

In another preferred embodiment a patient with cancer metastatic to a lumbar vertebrae complaining of severe persistent pain is treated by injection of osteoprotegerin in a therapeutically effective dose to the anatomic area adjacent to the involved vertebrae.

In another preferred embodiment injection of the therapeutic molecule to the anatomic area adjacent to the spinal metastasis is accomplished by interspinous injection.

In another preferred embodiment interspinous injection is accomplished by injection through the skin and through the interspinous ligament, either immediately above or immediately below the site of spine metastasis.

An example of one preferred embodiment for treatment of a breast cancer metastasis to the fourth lumbar vertebrae is the perilesional injection of etanercept 25 mg by injecting through the skin of the back, carried through the interspinous ligament at either the L3-L4 interspace or at the L4-L5 interspace, to deliver etanercept in anatomic proximity to the site of bone metastasis.

In another preferred embodiment injection of the therapeutic molecule to the anatomic area adjacent to the spinal metastasis is accomplished by subcutaneous injection.

In another preferred embodiment injection of the therapeutic molecule to the anatomic area adjacent to the spinal metastasis is accomplished by epidural injection.

In another preferred embodiment injection of the therapeutic molecule to the anatomic area adjacent to the spinal metastasis is accomplished by peridural injection.

In another preferred embodiment injection of the therapeutic molecule to the anatomic area adjacent to the spinal metastasis is accomplished by perispinal injection.

### Scientific Background

Antibodies (immunoglobulins) are proteins produced by one class of lymphocytes (B cells) in response to specific exogenous foreign molecules (antigens). Monoclonal antibodies (mAB), identical immunoglobulin copies which recognize a single antigen, are derived from clones (identical copies) of a single B cell. This technology enables large quantities of an immunoglobulin with a specific target to be mass produced.

Monoclonal antibodies with a high affinity for a specific cytokine will tend to reduce the biologic activity of that cytokine. Substances which reduce the biologic effect of a cytokine can be described in any of the following ways: as a cytokine blocker; as a cytokine inhibitor; or as a cytokine antagonist. In this patent, the terms blocker, inhibitor, and antagonist are used interchangeably with respect to cytokines.

Advances in biotechnology have resulted in improved molecules as compared to simply using monoclonal antibodies. One such molecule is CDP 870 which, rather than being a monoclonal antibody, is a new type of molecule, that being an antibody fragment. By removing part of the antibody structure, the function of this molecule is changed so that it acts differently in the human body. Another new type of molecule, distinct from monoclonal antibodies and soluble receptors, is a fusion protein. One such example is etanercept. This molecule has a distinct function which acts differently in the human body than a simple soluble receptor or receptors.

Monoclonal antibodies, fusion proteins, OPG, and all of the specific molecules discussed above under the categories of TNF antagonists and interleukin antagonists are considered biologics, in contrast to drugs that are chemically synthesized. These biologics are derived from living sources (such as mammals (including humans), other animals, and microorganisms). The biologics mentioned above are manufactured using biotechnology, which usually involves the use of recombinant DNA technology. Cytokine antagonists are one type of biologic. Biologics are regulated through a specific division of the FDA.

Cytokine antagonists can take several forms. They may be monoclonal antibodies (defined above). They may be a monoclonal antibody fragment. They may take the form of a soluble receptor to that cytokine. Soluble receptors freely circulate in the body. When they encounter their target cytokine they bind to it, effectively inactivating the cytokine, since the cytokine is then no longer able to bind with its biologic target in the body. An even more potent antagonist consists of two soluble receptors fused together to a specific portion of an immunoglobulin molecule (Fc fragment). This produces a dimer composed of two soluble receptors which have a high affinity for the target, and a prolonged half-life. This new molecule is called a fusion protein. An example of this new type of molecule, called a fusion protein, is etanercept (Enbrel).

Tumor necrosis factor (TNF), a naturally occurring cytokine present in humans and other mammals, plays a key role in the inflammatory response, in the immune response, in the response to infection, and is also involved in the promotion of osteoclastogenesis. TNF is formed by the cleavage of a precursor transmembrane protein, forming soluble molecules which aggregate in vivo to form trimolecular complexes. These complexes then bind to receptors found on a variety of cells. Binding produces an array of pro-inflammatory effects, including release of other pro-inflammatory cytokines, including IL-6, IL-8, and IL-1; release of matrix metalloproteinases; and up regulation of the expression of endothelial adhesion molecules, further amplifying the inflammatory and immune cascade by attracting leukocytes into extravascular tissues.

Interleukin-1 is a naturally occurring cytokine, present in humans and other mammals. Interleukin-1 plays a key role in the inflammatory response, in the immune response, and is also involved in the promotion of osteoclastogenesis. Interleukin-1 receptor antagonist (IL-1 RA) is a naturally occurring molecule which reduces the biologic effects of interleukin-1 by interfering with the binding of IL-1 to its receptor (IL-1 R1, interleukin-1 type 1 receptor). Kineret® (Amgen) is a recombinant form of IL-1 RA which is FDA approved for treating rheumatoid arthritis. It inhibits destruction of bone in patients with arthritis at least in part by interfering with immune-mediated osteoclastogenesis. IL-1 Receptor Type 2 (Amgen), AMG719 (Amgen), and IL-1 Trap (Regeneron) are all biologic inhibitors of interleukin-1.

Osteoprotegerin (OPG) is a biologic which is a potent inhibitor of bone resorption in vivo. It acts as a decoy receptor, binding and inactivating OPG Ligand (OPGL), which is an essential factor required for osteoclast differentiation.

Certain malignancies, such as breast cancer, have a proclivity to metastasize to bone and induce bone destruction, apparently through stimulation of osteoclasts[1,2]. Recent studies have suggested that TNF alpha leads to osteoclast activation and therefore may lead to bone destruction[3,4]. Interference with osteoclast activation is thought to be one of the mechanisms by which TNF inhibition leads to reduced bone destruction in patients with arthritis. The use of TNF antagonists to treat patients with bone metastases is an invention of the author.

Etanercept (Enbrel®, Amgen) is a potent and selective inhibitor of TNF. It is approved for chronic systemic use by subcutaneous injection to treat a variety of systemic inflammatory disorders, including rheumatoid arthritis. Multicenter trials in patients with rheumatoid arthritis, psoriatic arthritis, and ankylosing spondylitis have documented the ability of etanercept to reduce tissue damage, including the appearance of new bone lesions in these patients[5,6,7].

### Case Reports

Based on these known clinical results and the potential for etanercept to interfere with TNF-mediated bone destruction, etanercept was administered to two patients with treatment-refractory pain due to cancer metastasis to the spine.

Patient 1.—A 77 y.o. Caucasian woman began having severe and unrelenting mid-back pain in January 2001. In May 2001 she developed a dry, unproductive cough. In July 2001 a rapidly growing nodule appeared on the right forehead. Biopsy showed adenocarcinoma with papillary features, TTF-1 immunoperoxidase stain positive. Chest x-ray revealed a mass in the right lung interpreted to be the primary carcinoma. CT imaging of the lung in August 2001 showed a 5 cm. mass in the right lower lobe of the lung. Bone scan in August 2001 showed intense focal areas of increased uptake involving the right frontal skull and spine at T11. Plain x-rays of the back showed a metastatic lesion of the T11 vertebrae, with compression fracture and destruction of one pedicle. The patient was treated with two courses of radiation to her right forehead lesion, and a single course of radiation to T11 in September, but severe, unremitting back pain continued. The patient was prescribed fentanyl patches and morphine, which she required daily throughout August and September, but pain control was poor. The patient had difficulty ambulating due to the pain, and could not exit or enter an automobile without assistance. In October, ten months after back pain began, the patient presented to our office for treatment of the localized mid-back pain which was constant, present 24 hours per day, and despite multiple daily doses of morphine was not adequately controlled. After informed consent was obtained, etanercept (Enbrel®, Immunex) 25 mg was administered subcutaneously to the mid-back.

Patient 2—A 50 y.o. Caucasian woman was well until seven years previously when infiltrating ductal carcinoma of the right breast was diagnosed, ER negative and node negative. She was treated with lumpectomy and radiation therapy. Four years later she developed bilateral hip pain and several months later low back pain began. Bone scan showed increased uptake in the ischium, ribs, and lumbar spine at L4. The patient underwent left hip reconstruction and replacement due to metastatic disease. Biopsy specimens from the hip surgery yielded tissue which was characterized as ER+ and 3+Her-2 positive. Treatment with trastuzumab, biphosphonates, and strontium-89 was begun. The patient required daily narcotics for pain, but control was inadequate. One month prior to her visit to our office a PET scan documented increased metabolic activity in the lower lumbar spine at L4 and in a lower right rib, consistent with bone metastases. The patient presented to our office for treatment of intractable low back pain of two years duration and was able to ambulate only with the use of crutches due to pathologic fractures involving both hips. The low back pain at the site of spinal metastasis at L4 was constant, present 24 hours per day for more than two years. Informed consent was obtained. Etanercept (Enbrel®, Immunex) 25 mg by subcutaneous injection was administered to the lumbar area.

Results of Case Treatments

Treatment with etanercept, administered by subcutaneous injection, resulted in rapid, substantial, and prolonged relief of previously treatment-refractory pain in each of these patients with cancer metastatic to their spine.

Patient 1 reported substantial pain relief within ten minutes of etanercept administration. Within 24 hours following administration of etanercept she became completely pain-free. The multiple doses of morphine which had been necessary for two months prior to etanercept were no longer required. In addition she reported an improved appetite, and the ability to get into or out of a car without assistance. At one month complete relief of back pain continued. At five weeks moderately severe mid-back pain returned, accompanied by difficulty rising out of a chair without assistance, and a second dose of etanercept 25 mg subcutaneously was administered. One day after the second dose pain relief began. After two days relief of back pain was complete, and she experienced no further back pain for the rest of her life, which lasted an additional five months until death ensued from extension of the right forehead lesion to the brain.

Patient 2 experienced rapid relief of spinal pain, which she reported as 90% improvement in pain within five minutes, and 95% improvement lasting from one day to three weeks following treatment. At five weeks following the single dose of etanercept the patient continued to report 90% relief of her lower back pain. Immediately following treatment the patient was able to decrease her pain medication significantly. Oswestry Pain Disability score[8]prior to treatment was 58%; at 11 days following treatment it was 33%; at 14 days following treatment the score improved to 28%, and continued to be 28% at 21 days. At 35 days post-treatment the Oswestry score was 33%.

Discussion of Case Reports

Tumor metastasis to bone is a common clinical event, often leading to intractable pain and bone destruction, both of which are often refractory to treatment [9]. Remodeling of the skeleton is a constant process requiring a delicate balance between bone formation, mediated by osteoblasts, and bone destruction, mediated by osteoclasts[10,11]. Increased bone destruction mediated by osteoclasts has been reported in breast cancer metastasis in animals[2]. In an experimental model with human breast cancer cells it was shown that release of TNF alpha by breast cancer cells was responsible for stimulating osteoclast fusion and migration, thereby stimulating osteoclast-mediated bone resorption [12]. Other studies support the concept that destruction of bone by breast cancer metastases is mediated by osteoclasts [13].

The inflammatory cytokine TNF alpha has been shown to promote osteoclast activity through several mechanisms, including induction of osteoclast differentiation and indirectly via the induction of RANKL/RANK signaling [14-18]. Inhibition of TNF alpha, by interfering with TNF mediated osteoclastogenesis, reduces bone destruction in patients with osteolytic malignant bone metastases.

A new group of biologic medications which modulate the action of the inflammatory cytokine Tumor Necrosis Factor alpha (TNF) have recently become available for therapeutic use. These agents are potent and selective inhibitors of TNF in the human. Their principal uses have been to treat Rheumatoid Arthritis in adults [19,20], juvenile rheumatoid arthritis in children [21], and Crohn's Disease. The currently FDA-approved TNF antagonists of biologic origin are etanercept (Enbrel®, Immunex) and infliximab (Remicade®, Johnson and Johnson). In addition, D2E7 (Abbott) and CDP 870 (Celltech) are in clinical development. Several other orally administered drugs have significant TNF antagonist activity, the most important of these being thalidomide. TNF antagonists, administered parenterally, are a new treatment modality for bone metastases. The methods of administration of these agents to treat bone metastases include systemic administration, including subcutaneous, intramuscular, or intravenous routes; or localized anatomic administration, including intralesional or perilesional administration.

Other cytokines, including interleukin-1, M-CSF, and OPG are involved in the mediation of bone metabolism [22,23]. These agents, or their specific inhibitors, such as IL1-RA, IL1-Receptor Type 2, and IL-1 Trap (Regeneron) may also prove to have clinical benefit due to their biologic effects on osteoclastogenesis. It has been suggested that inhibition of osteoclasts at different levels of activity may be therapeutically useful, such as through the combination of OPG and biphosphonates[3]. Other combinations may show additive or even synergistic clinical benefit. Included among these combinations would be any combination of the above mentioned therapeutic agents, including, but not limited to the following: OPG and TNF inhibition; OPG and IL1-RA; and etanercept and IL1-RA, both given by localized perilesional administration to minimize systemic effects and maximize local therapeutic efficacy.

### Dosages and Routes of Administration

The dosage of a cytokine antagonist used for intralesional or perilesional administration will in general be within one order of magnitude of the dosage used as a single dose for systemic administration. For example, if the usual dose when administered systemically is 100 mg, then the dose used for intralesional therapy will usually be between 10 mg and 100 mg. One exception to this rule is the dose for administration into an anatomically confined structure. In this case, if the structure is small, the dose will need to be reduced accordingly.

For the treatment of acute or severe conditions, the dose will generally be adjusted upward. In the above example the dose selected would therefore be 100 mg, rather than 10 mg, if the condition were acute and/or severe.

Localized perilesional injection can allow the use of subcutaneous administration even in the case when the medication is normally administered intravenously. An example of this would be the use of infliximab subcutaneously to an anatomically adjacent area for the treatment of a malignant bone metastasis.

For treating the above diseases with the above mentioned TNF antagonists, these TNF antagonists may be administered by the following routes:

The above TNF antagonists may be administered subcutaneously in the human and the dosage level is in the range of 1 mg to 300 mg per dose, with dosage intervals as short as two days.

The above TNF antagonists may be administered intramuscularly in the human and the dosage level is in the range of 1 mg to 200 mg per dose, with dosage intervals as short as two days.

The above TNF antagonists may be administered epidurally in the human and the dosage level is in the range of 1 mg to 300 mg per dose, with dosage intervals as short as two days.

The above TNF antagonists may be administered peridurally in the human and the dosage level is in the range of 1 mg to 300 mg per dose, with dosage intervals as short as two days.

The above TNF antagonists may be administered by interspinous injection in the human and the dosage level is in the range of 1 mg to 300 mg per dose, with dosage intervals as short as two days.

Interleukin-1 antagonists are administered in a therapeutically effective dose, which will generally be 25% to 100% of the usual dose administered for treatment of rheumatoid arthritis. The dosage interval will be the same or less often than that used to treat rheumatoid arthritis.

Osteoprotegerin is administered in a therapeutically effective dose.

### Advantages of the Present Invention

Accordingly, an advantage of the present invention is that it provides for the localized administration of specific biologics as a new pharmacologic treatment of malignant bone metastases; such that the use of these biologics will result in the amelioration of these conditions.

Another advantage of the present invention is that it provides for specific biologics delivered by anatomically localized administration, which, when compared to systemic administration, produces one or more of the following: greater efficacy; more rapid onset; longer duration of action; or fewer side effects.

Another advantage of the present invention is that it provides for specific biologics for providing suppression and inhibition of the action of cytokines in a human to treat malignant bone metastases.

Another advantage of the present invention is that it provides for specific biologics administered by specific methods for treating humans with a malignant bone metastasis or metastases which due to their biologic action will produce clinical improvement in the patient and will give the patient a better opportunity to heal, slow disease progression, prevent neurological damage, reduce pain, or otherwise improves the patient's health.

Another advantage of the present invention is that it provides for specific biologics, including cytokine antagonists to tumor necrosis factor alpha or to interleukin-1, using localized administration, including perilesional or intralesional administration, as the preferred form of administration, for the treatment of malignant bone metastases.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

### REFERENCES

[1]. Yoneda T, Sasaki A, Mundy G R. Osteolytic bone metastasis in breast cancer. Breast Cancer Res Treat (1994);32:73-84.

[2]. Clohisy D R, Ogilvie C M, Carpenter R J, Ramnaraine M L R. Localized tumor-associated osteolysis involves the recruitment and activation of osteoclasts. J Orthop Res (1996);14:2-6.

[3]. Redlich K, Hayer S, Maier A, Dunstan C R, Tohidast-Akrad M, Lang S, Turk B, Pietschmann P, Woloszczuk W, Haralambous S, Kollias G, Steiner G, Smolen J S, Schett G. Tumor necrosis factor alpha-mediated joint destruction is inhibited by targeting osteoclasts with osteoprotegerin. Arthritis Rheum. (2002)March; 46(3):785-92.

[4]. Chu C Q, Field M, Feldmann M, Maini R N. Localization of tumor necrosis factor in synovial tissues and at the cartilage-pannus junction in patients with rheumatoid arthritis. Arthritis Rheum (1991);34:1125-32.

[5]. Bathon J M, Martin R W, Fleischmann R M, et al. A comparison of etanercept and methotrexate in patients with early rheumatoid arthritis. N Engl J Med (2000);343:1586-1593.

[6]. Mease P J, Goffe B S, Metz J, VanderStoep A, Finck B, Burge D J. Etanercept in the treatment of psoriatic arthritis and psoriasis: a randomised trial. Lancet. (2000) July; 29;356(9227):385-90.

[7]. Gorman J D, Sack K E, David J C. Treatment of ankylosing spondylitis by inhibition of tumor necrosis factor-alpha. N Engl J Med (2000);346:1349-1 356.

[8]. Fairbank J, Davies J, Coupar J, O'Brien J P. The Oswestry low back pain disability questionnaire. Physiotherapy (1980);66:271-3.

[9]. Janjan N. Bone metastases: approaches to management. Semin Oncol. (2001) August;28(4 Suppl 11):28-34.

[10]. Pacifici R. Estrogen, cytokines, and pathogenesis of postmenopausal osteoporosis. J Bone Miner Res (1996);11:1043-1051

[11]. Manolagas S C. Birth and death of bone cells: basic regulatory mechanisms and implications for the pathogenesis and treatment of osteoporosis. Endocr Rcv (2000);21:115-137.

[12]. Tumber A, Morgan H M, Meikle M C, Hill P A. Human breast-cancer cells stimulate the fusion, migration and resorptive activity of osteoclasts in bone explants. Int. J. Cancer (2001);91:665-672.

[13]. Clohisy D R, Palkert D, Ramnaraine M L R, Pekurovsky I, Oursler M J. Human breast cancer induces osteoclast activation and increases the number of osteoclasts at sites of tumor osteolysis. J Orthop Res (1996);14:396-402.

[14]. Van der Pluijm G, Most W, Van der Wee-Pals L, de Groot H, Papapoulos S, Lowik C. Two distinct effects of recombinant human tumour necrosis factor-a on osteoclast development and subsequent resorption of mineralized matrix. Endocrinology (1991);129:1596-604.

[15]. Azuma Y, Kaji K, Katogi R, Takeshita S, Kudo A. Tumor necrosis factor-alpha induces differentiation of and bone resorption by osteoclasts. J Biol Chem (2000);275:4858-64.

[16]. Kobayashi K, Takahashi N, Jimi E, Udagawa N, Takami M, Kotake S, et al. Tumor necrosis factor alpha stimulates osteoclast differentiation by a mechanism independent of the ODF/RANKLRANK interaction. J Exp Med (2000);191:275-86.

[17]. Abu-Amer Y, Erdmann J, Alexopoulou L, Kollias G, Ross F P, Teitelbaum S L. Tumor necrosis factor receptors types 1 and 2 differentially regulate osteoclastogenesis. J Biol Chem (2000);275:27307-10.

[18]. Suda T, Kobayashi K, Jimi E, Udagawa N, Takahashi N. The molecular basis of osteoclast differentiation and activation. Novartis Found Symp (2001);232:235-50.

[19]. Moreland L W, Schiff M H, Baumgarmer S W, et al. Etanercept therapy in rheumatoid arthritis: a randomized, controlled trial. N Engl J Med (1999);130:478-486.

[20]. Weinblatt M E, Kremer J M, Bankhurst A D, et al. A trial of etanercept, a recombinant tumor necrosis factor receptor:Fc Fusion protein, in patients with rheumatoid arthritis receiving methotrexate. N Engl J Med (1999);340(4):253-259.

[21]. Lovell D J, Giannini E H, Reiff A, et al. Etanercept in children with polyarticular juvenile rheumatoid arthritis. N Engl J Med (2000);342:763-769.

[22]. Jimi E, Nakamura I, Duong L T, Ikebe T, Takahashi N, Rodan G A, et al. Interleukin 1 induces multinucleation and bone resorbing activity of osteoclasts in the absence of osteoblasts/stromal cells. Exp Cell Res (1999);247:84-93.

[23]. Hofbauer L C, et al. The roles of osteoprotegerin and osteoprotegerin ligand in the paracrine regulation of bone resorption. J Bone Miner Res (2000);15:2-12.

What is claimed is:

1. A method for treating malignant bone metastases in a human by inhibiting the action of tumor necrosis factor (TNF) through the administration of a TNF antagonist comprising the steps of:
a) administering a therapeutically effective dosage level to said human of said TNF antagonist selected from the group consisting of etanercept, infliximab, CDP571 (a humanized monoclonal anti-TNF-alpha IgG4 antibody), CDP 870 (a humanized monoclonal anti-TNF-alpha antibody fragment), D2E7 (a human anti-TNF mAb), soluble TNF receptor Type I, pegylated soluble TNF receptor Type I (PEGs TNF-R1) and onercept, a recombinant TNF binding protein (r-TBP-1) (Serono); and
b) administering said dose parenterally by perilesional injection to the area anatomically adjacent to the site of malignant bone metastasis.

2. A method for treating malignant bone metastases in a human by inhibiting the action of tumor necrosis factor (TNF) through the administration of infliximab comprising the steps of:
a) administering a therapeutically effective dosage level to said human of said infliximab; and
b) administering said dose parenterally to the area anatomically adjacent to the site of malignant bone metastasis.

3. A method for treating malignant bone metastases in a human in accordance with claim 2, wherein the step of administering said dose parenterally is by perilesional or intralesional injection.

4. A method for treating malignant bone metastases in a human by inhibiting the action of tumor necrosis factor (TNF) through the administration of onercept comprising the steps of:
a) administering a therapeutically effective dosage level to said human of said onercept; and
b) administering said dose parenterally to the area anatomically adjacent to the site of malignant bone metastasis.

5. A method for treating malignant bone metastases in a human in accordance with claim 4, wherein the step of administering said dose parenterally is by perilesional or intralesional injection.

6. A method for treating malignant bone metastases in a human by inhibiting the action of tumor necrosis factor (TNF) through the administration of infliximab comprising the steps of:
a) administering a therapeutically effective dosage level to said human of said infliximab; and
b) administering said dose parenterally.

7. A method for treating malignant bone metastases in a human in accordance with claim 6, wherein the step of administering said dose parenterally is by perilesional or intralesional injection.

8. A method for inhibiting the action of TNF in accordance with claim 1, wherein the step of administering said TNF antagonist in the form of etanercept is performed parenterally in said human wherein said dosage level is in the range of 2 mg to 100 mg per dose.

9. A method for inhibiting the action of TNF in accordance with claim 1, wherein the step of administering said TNF antagonist in the form of infliximab is performed parenterally in said human wherein said dosage level is in the range of 1 mg to 500 mg per dose.

10. A method for inhibiting the action of TNF in accordance with claim 1, wherein the step of administering said TNF antagonist in the form of D2E7 is performed parenterally in said human wherein said dosage level is in the range of 1 mg to 200 mg per dose.

11. A method for inhibiting the action of TNF in accordance with claim 1, wherein the step of administering said TNF antagonist in the form of CDP 870 is performed parenterally in said human wherein said dosage level is in the range of 1 mg to 300 mg per dose.

12. A method for treating malignant bone metastases in a human by inhibiting the action of interleukin-1 (IL-1) through the administration of a IL-1 antagonist comprising the steps of:
a) administering a therapeutically effective dosage level to said human of said IL-1 antagonist and,
b) administering said dose either intralesionally or perilesionally.

13. A method for inhibiting the action of IL-1 in accordance with claim 12, wherein the step of administering said dosage level is for treating cancer metastatic to bone.

14. A method for inhibiting the action of IL-1 in accordance with claim 12, wherein the step of administering said dosage level is for treating spinal cord compression due to cancer metastatic to bone.

15. A method for inhibiting the action of IL-1 in accordance with claim 12, wherein the step of administering said IL-1 antagonist is performed through any of the following routes: subcutaneous, intramuscular, interspinous, peridural, parenteral, perispinal, or epidural.

16. A method for treating malignant bone metastases in a human by inhibiting the action of interleukin-1 (IL-1 ) through the administration of a IL-1 antagonist comprising the steps of:
a) administering a therapeutically effective dosage level to said human of said IL-1 antagonist selected from the group consisting of IL-1 receptor antagonist; Kineret® (Amgen); IL-1 Receptor type 2 (Amgen); AMG719 (Amgen) and IL-1 Trap (Regeneron); and
b) administering said dose parenterally by perilesional injection to the area anatomically adjacent to the site of malignant bone metastasis.

17. A method for treating malignant bone metastases in a human by administering osteoprotegerin (OPG) comprising the steps of:
a) administering a therapeutically effective dosage level to said human of said OPG and,
b) administering said dose either intralesionally or perilesionally.

18. A method for treating malignant bone metastases in a human in accordance with claim 17, wherein the step of administering said OPG is performed through any of the following routes: subcutaneous, intramuscular, interspinous, peridural, parenteral, perispinal, or epidural.

19. A method for treating malignant bone metastases in a human by inhibiting the action of tumor necrosis factor (TNF) through the administration of a TNF antagonist comprising the steps of:
a) administering a therapeutically effective dosage level to said human of said TNF antagonist; and,
b) administering said dose parenterally.

20. A method for inhibiting the action of TNF in accordance with claim 19, wherein the step of administering said dosage level is for treating cancer metastatic to bone.

21. A method for inhibiting the action of TNF in accordance with claim 19, wherein the step of administering said dosage level is for treating spinal cord compression due to cancer metastatic to bone.

22. A method for inhibiting the action of TNF in accordance with claim 19, wherein the step of administering said TNF antagonist is performed through any of the following routes: subcutaneous, intramuscular, intravenous, interspinous, peridural, parenteral, perispinal, or epidural.
